(19) United States
(12) Patent Application Publication
(10) Pub. No.: US 2006/0183681 A1
(43) Pub. Date: Aug. 17, 2006

(54) Stabilized compositions containing natriuretic peptides

(75) Inventors: Alireza Ebrahim, Laguna Niguel, CA (US); Timothy Ho, Foothill Ranch, CA (US); James Cole, Yorba Linda, CA (US)

Correspondence Address: TOWNSEND AND TOWNSEND AND CREW, LLP, TWO EMBARCADERO CENTER, EIGHTH FLOOR, SAN FRANCISCO, CA, 94111-3834, US

(73) Assignee: Bio-Rad Laboratories, Inc., Hercules, CA (US)

(21) Appl. No.: 11/058,465

(22) Filed: Feb. 14, 2005

(51) Int. Cl. A61K 38/17

(52) U.S. Cl. 514/12

(57) ABSTRACT

Stabilized compositions of natriuretic peptides comprise the peptide and an effective stabilizing amount of (i) an alkyl or aryl sulfonyl fluoride protease inhibitor, or (ii) benzamidine:

### BACKGROUND OF THE INVENTION

This invention relates to stable compositions of natriuretic peptides, notably B-type natriuretic peptide (BNP) in general, and for use as control materials, for example for monitoring of the performance of the BNP test procedures for this biochemical marker that are used for diagnosis and staging of patients with congestive heart failure (CHF). The invention also relates to the preparation of such compositions.

Heart failure is a complex clinical syndrome resulting from a cardiac disease, compromising ventricular systolic or diastolic function, or both. It results from an inability of the heart to pump blood at a sufficient level to supply the oxygen and metabolic needs of the body. Congestive heart failure is a clinical condition in which the heart is unable to supply the body with enough oxygen-rich blood to accommodate the body's needs. As a result of the decreased cardiac function, body fluids may accumulate in the lungs and peripheral vascular space. The most common cause of CHF is ischemic heart disease. Other causes of CHF are hypertension, myocarditis, and valvular disease.

Natriuretic peptides are a class of hormones that regulate blood pressure, electrolyte balance, and fluid volume. Atrial natriuretic peptide (ANP) is a 28-amino acid hormone that originates from the atria of the heart. B-type natriuretic peptide (originally referred to as “brain natriuretic peptide”) is a 32-amino acid hormone that is secreted from the ventricles. Within the myocyte, BNP is derived from prepro BNP (a 134-amino acid peptide), which is. cleaved to proBNP (a 108-amino acid peptide) and another 26-amino acid peptide. Other natriuretic peptides are C-type natriuretic peptide (CNP) and Dendroaspis natriuretic peptide (DNP). ANP and BNP belong to the cardiac natriuretic system, are of myocardial cell origin and share a wide spectrum of biological properties. CNP is of endothelial cell origin; it is found in the brain and cerebrospinal fluid; however, little if any is present in the heart. DNP was isolated from the venom of the green mamba snake, and possesses structural similarity to ANP, BNP and CNP. DNP-like immunoreactivity has been found to be elevated in patients with congestive heart failure.

Both natural and synthetic natriuretic peptides, and their derivatives, are well known, as are methods for preparation of synthetic natriuretic peptides.

Plasma concentrations of the fragments of pro BNP [BNP and N-terminal BNP (NT pro BNP)] are increased in patients with CHF and have been shown to accurately predict clinical severity and left ventricular ejection fraction as well as morbidity and mortality in patients. In recent years, this indicator of CHF disease severity has been used to diagnose and classify the severity of the congestive heart failure. According to the New York Heart Association classification of CHF, the mean concentrations of BNP progressively increase from stage I to IV. In a multi-center clinical trial, mean BNP concentrations of 71 pg/ml, 204 pg/ml, 349 pg/ml, and 1022 pg/ml were observed for stages I, II, II, and IV of congestive heart failure, respectively. Stage IV of CHF represents the highest severity of the disease and is defined as the cardiac disease resulting in inability to carry on any physical activity without discomfort. Patient in this stage of the disease may have symptoms of heart disease or the coronary syndrome even at rest. Furthermore, the level of discomfort in these patients will increase if any physical activity is undertaken.

A number of diagnostic tests for BNP using different technologies have been described in the literature and introduced to the clinical laboratory market. The Abbott AxSYM®, Bayer ADVIA Centaur®, and Biosite Triage® BNP assays are some of the quantitative test methods available in the market for determination of BNP. The Abbott AxSYM assay utilizes the Microparticle Enzyme Immunoassay (MEIA) technology, which uses microparticles coated with anti-BNP monoclonal antibodies that bind to human BNP antigen. These antigen-antibody complexes on the microparticles are later treated with another monoclonal anti-BNP alkaline phosphatase conjugate. The final complex will then catalyze the removal of a phosphate group from a fluorescent substrate, yielding a fluorescent product. The fluorescent intensity of the product will then be measured by the optical assembly to determine the concentration of BNP. The Biosite Triage BNP assay is an immunofluorometric assay. In this assay, a murine recombinant polyclonal antibody is bound to the fluorescent label, and a murine monoclonal antibody against the disulfide bond-mediated ring structure of BNP is bound to the solid phase. In this assay, plasma is allowed to react with fluorescent antibody conjugates. After an incubation period, complexes of BNP and the fluorescent antibody conjugate are captured on a detection lane. The concentration of BNP in the specimen, which is proportional to the fluorescence bound to the detection lane, is then determined quantitatively by a handheld fluorescence instrument. The Bayer ADVIA Centaur assay is a two-site sandwich immunoassay using chemiluminescent technology. The first antibody used in this assay is an acridinium ester labeled monoclonal mouse anti-human BNP specific to the ring structure on BNP. The second antibody (solid phase) is a biotinylated monoclonal mouse anti-human antibody specific to the C-terminal portion of BNP, which is coupled to streptavidin magnetic particles. The limits of detection for the Abbott AxSYM, Biosite Triage, and Bayer ADVIA Centaur BNP assays are 15, 5, and 2 pg/mL, respectively.

Quality control materials are routinely used in clinical diagnostics laboratories to monitor the precision and accuracy of the clinical test methods and procedures. The quality control material should be as sensitive as the actual patient sample to all of the anticipated analytical variances. Furthermore, the quality control material should be stable, and its analyte target concentrations should challenge the medical decision point of the assay. Other desirable features of a quality control material are low cost, lot-to-lot reproducibility, and ease of manufacturing.

Several BNP controls are currently available in the market including those from instrument manufacturers, Abbott and Bayer. The Abbott BNP Control (REF 8G82-10) is a tri-level liquid control composed of BNP in an acetate buffer with bovine protein stabilizers and preservatives sodium azide and ProClin 300. Some assays of this general type are described in U.S. published patent applications 2005/0014287 (Friese et al.) and 2005/0014289 (Parsons et al.). The Bayer BNP Control (REF 02817045) is a tri-level lyophilized control comprised of synthetic human BNP in buffered sodium caseinate with sodium azide.

BNP controls are typically manufactured using artificial and buffered matrices instead of human serum or plasma because of the poor stability of this peptide in serum or plasma. The half-life (t_{1/2}) of BNP in vivo is approximately 23 minutes. Even in these artificial matrices, stability of BNP is not very long. For example, BNP is only stable in the Bayer BNP Control (buffered sodium caseinate) for 5 days when reconstituted and stored at 2-8° C.

BNP is cleared from circulation by specific cellular receptors and endopeptidases. The main reason for poor stability of BNP could be attributed to the presence of natural proteases in plasma or serum. Several approaches may be used to protect this peptide from oxidative and enzymatic degradation for the purpose of manufacturing stable BNP controls, for example:
1) Derivatization of BNP to make it unsuitable as a substrate for catalytic sites of the proteases
2) Use of high molecular weight molecules to protect BNP by providing, a caging effect
3) Use of heat to inactivate natural proteases present in serum or plasma
4) Use of a non-specific substrate to compete with BNP for catalytic sites on the proteases
5) Use of reducing agent(s)
6) Use of potent and specific inhibitor(s) to eliminate or minimize catalytic activity of proteases (specific inhibitors that have been so used include EDTA, which is a reversible inhibitor of metalloproteases, and aprotinin, which is an inhibitor of a number of serine proteases)

Most of the above approaches appear to be ineffective, expensive, and/or require dedicated and custom equipment and vessels, and may result in denaturation of protein, increased turbidity, and interference with the analytical signal used in the immunoassay. Identification of the proteases and silencing specific proteases with a few protease inhibitors as well as the use of appropriate reducing agents appear to have many advantages over the other approaches mentioned above, when trying to stabilize BNP in liquid human serum.

Therefore, there exists a need for a stable and serum-based quality control material for use with BNP and other natriuretic peptide assays. There also exists a need for stable compositions of these peptides in general, for any suitable use, for example, in conducting studies of the properties and/or behavior of natriuretic peptides. The present invention satisfies these needs and meets other essential requirements for a quality control material, such as responding in the same way to analytical variances as a patient sample by using human or other mammalian serum or plasma as the base matrix, having target values that challenge the linear dynamic range of the assay, and providing acceptable, open vial and closed vial stabilities for long term use.

### SUMMARY OF THE INVENTION

In general, this invention comprises stabilized cormpositions containing or comprising endogenous or exogenous natriuretic peptides (native, synthetic, or recombinant). More specifically, the invention comprises such compositions also comprising mammalian, including human, plasma or serum, especially human plasma or serum, and more particularly, processed human plasma. Still more specifically the invention comprises stabilized compositions containing or comprising natriuretic peptides and one or more optionally substituted alkyl or aryl sulfonyl fluoride protease inhibitors, or benzamidine. Such compositions may be used, for instance, for preparing reference materials to monitor the performance of various clinical test methods using BNP or other natriuretic peptides. The compositions may also be prepared for other uses of stabilized natriuretic peptide compositions such as conducting studies of the properties or behavior of natriuretic peptides.

In another aspect the invention comprises kits for assaying for a natriuretic peptide comprising such a control composition. In yet another aspect, the invention comprises methods for preparing such stabilized compositions.

### BRIEF DESCRIPTION OF THE DRAWINGS

FIG. 1 depicts open-vial stability of BNP in the presence and absence of proteases inhibitors

FIG. 2 depicts open-vial stability of a tri-level BNP control.

### DETAILED DESCRIPTION OF THE INVENTION

As stated above, in general, this invention comprises stabilized compositions containing or comprising endogenous or exogenous natriuretic peptides (native, synthetic, or recombinant). More specifically, the invention comprises such compositions also comprising human or other mammalian plasma or serum, particularly processed plasma. Still more specifically the invention comprises stabilized compositions containing or comprising natriuretic peptides and one or more specific protease inhibitors as described herein. Such compositions may be used, for instance, for preparing reference materials to monitor the performance of various clinical test methods using BNP or other natriuretic peptides. The compositions may also be prepared for other uses of stabilized natriuretic peptide compositions.

In another aspect the invention comprises methods for preparing such compositions.

As used herein, the terms “natriuretic peptide” and “natriuretic peptides” include such peptides in general, particularly ANP, BNP, CNP and DNP, as well as precursors of such peptides such as pro- and prepro-peptides, for example proBNP and preproBNP described above. This term includes such substances whether exogenous or endogenous, whether existing naturally, or synthesized, or prepared using recombinant DNA techniques.

In accordance with this invention, it has now been determined that certain optionally substituted alkyl and aryl sulfonyl fluorides and benzamidine are suitable protease inhibitors for use in the compositions of this invention. Phenylmethylsulfonylfluoride (PMSF) and 4-amidinophenyl-methaniesulfonyl fluoride (APMSF) have been used to inhibit serine protease activity. These compounds react covalently with the serine residue at the catalytic site.

The alkyl and aryl sulfonyl fluorides suitable for use in the compositions, kits and methods of this invention, are those that inhibit proteolytic activities of trypsin, chymotrypsin, elastase, plasmin, thrombin, or kallikrein (using substrates such as labeled casein or other suitable peptide substrates).

The term “alkyl” as used herein means a straight or branched chain, or non-aromatic cyclical, hydrocarbon radical, or combination thereof, that is fully saturated and has the number of carbon atoms designated (i.e. C₁-C₁₀ means one to ten carbon atoms). Examples of acyclic alkyl groups include, but are not limited to, groups such as methyl, ethyl, n-propyl, isopropyl, n-butyl, t-butyl, isobutyl, sec-butyl, homologs and isomers of, for example, n-pentyl, n-hexyl, n-heptyl, n-octyl, and the like. Examples of cyclical alkyl groups include cyclopropyl, cyclobutyl, cyclopentyl, cyclohexyl, and the like. For use in the invention, alkyl groups generally may be of any desirable size. Preferably they will contain up to 8, more preferably, up to 4, carbon atoms.

The alkyl groups of compounds used in this invention may be unsubstituted or may be mono- or polysubstituted. Permissible substituents include those commonly found for such moieties, provided that they do not significantly interfere with the protease-inhibiting activity of the compound in question. Typical substituents include halo, hydroxy, amino, amido, nitro, cyano, alkoxy, oxo, and such substituents further containing optionally substituted alkyl groups such as alkylamino, haloalkylamino, haloalkoxy, and the like.

Substituted alkyl or cycloalkyl groups also include arylalkyl groups, namely alkyl (including cycloalkyl) groups substituted by one or more aryl groups; for instance, benzyl, phenethyl, triphenylmethyl, cyclohexylmethyl, cyclopropylmethyl, and the like. They also may include smaller cycloalkyl groups having an aryl group as a substituent such as phenylcyclopropyl. The aromatic ring or rings in the arylalkyl groups may be further substituted similarly to other aliphatic groups, e.g. chlorophenyl, methyl benzyl, etc. Substituted alkyl groups also include alkyl groups substituted by one or more saturated or unsaturated heterocyclic groups, e.g., pyridylmethyl, pyridylmethyl, piperidinylmethyl, pyrrolidinylmethyl, morpholinylmethyl, quinolylmethyl, etc. Such groups may be substituted by one or more halogens, hydroxyl groups, lower alkyl groups, or lower alkoxy groups (including combinations of such groups).

As used herein, “aryl” refers to the typical substituted or unsubstituted non-aliphatic hydrocarbyl groups of this class, i.e., a polyunsaturated, typically aromatic, hydrocarbon substituent, which can be a single ring or multiple rings (up to three rings) that are fused together or linked covalently, such as phenyl, naphthyl, and the like. This class of moieties also includes fused-ring moieties such as indanyl, etc. Substituents for the aromatic moieties are similar to those for the aliphatic groups. “Aryl”, as used herein, also includes analogous heterocyclic groups (sometimes termed “heteroaromatic” groups), namely polyunsaturated cyclical moieties containing carbon atoms in the ring and additionally one or more hetero atoms, which are typically oxygen, nitrogen, sulfur and or phosphorus, such as pyridinyl, pyrazinyl, pyrazolyl, thienyl, furyl, thiazolyl, imidazolyl, pyrrolyl, etc., and fused-ring moieties such as benzoxazolyl, benzthiazolyl, etc. These may be optionally substituted with one or more substituents such as halogen, hydroxy, amino, optionally substituted lower alkyl, optionally substituted acyl, optionally substituted lower alkoxy, alkyleneoxy, alkylenedioxy, optionally substituted arylacetamido, and the like.

The term “acyl” refers to a group derived from an organic acid by removal of the hydroxy group. Examples of acyl groups include acetyl, propionyl, dodecanoyl, tetradecanoyl, isobutyryl, and the like. Accordingly, the term “acyl” as used herein is meant to include a group otherwise defined as —C(O)-alkyl, where alkyl is as defined above.

The suitability of alkyl and aryl sulfonyl fluorides for use in this invention is based on their activity as protease inhibitors, as mentioned above, i.e. their capacity to eliminate or minimize the ability of the naturally occurring proteases in human serum or plasma to cleave natriuretic peptides, thereby resulting in degradation and poor stability of these peptides in these matrices. Candidate compounds whose activity as protease inhibitors is not known can be screened for use in the invention using a relatively simple assay in which the potential inhibitor is incubated with the enzyme(s)/protease(s) at certain temperature and certain period of time. To determine the inhibitory effect, a chromogenic substrate or BNP can be added to the protease-inhibitor mixture and the extent of cleavage or degradation determined using available separation and analytical methods (chromatography, spectrometry, immunoassay, etc.). Similar assays may be used to determine the minimum inhibitory concentration for a given sulfonyl fluoride that is found to be a suitable protease inhibitor.

A particular potent and irreversible protease inhibitor in the sulfonyl fluoride class, and thus a referred inhibitor for use in this invention, is (2-aminoethyl)-benzenesulfonyl fluoride (AEBSF, Formula: C₈H₁₀NO₂SF.HCl, Molecular Weight: 239.7). It shows negligible toxicity and broader inhibitory activity, and is only very slowly hydrolyzed under weak basic conditions (pH 8-9). Furthermore, after covalent bonding with the serine residue at the catalytic site, no hydrolysis back to the active protease is observed. Other advantages of AEBSF are good solubility in water and aqueous media and its selectivity e.g. the inhibitory activity related to thrombin activity is not delayed in the presence of serum albumin. Therefore, AEBSF is well suited for use in matrices such as serum or plasma.

Sulfonyl fluorides that are suitable for use in the compositions and methods of this invention include methanesulfonyl fluoride, phenylmethanesulfonyl fluoride (PMSF), 4-amidinophenyl-methanesulfonyl fluoride (APMSF), 3-acetylbenzenesulfonyl fluoride, 2 -aminobenzenesulfonyl fluoride, and 3-(3-chlorophenoxyacetamido)benzenesulfonyl fluoride. Peptide aminobenzene sulfonyl fluorides, i.e. benzenesulfonyl fluorides further substituted by a peptide chain, for example 2-[Ac-Ala-Ala-NHN(CH₃)CONH]C₆H₄SO₂F, also are suitable for use in the compositions and methods of the invention. Indeed, these inhibitors may increase the reactivity of the sulfonyl fluoride by adding an extended side chain that could provide some secondary binding interaction with the enzyme, with a subsequent increase in reaction rate.

Benzamidine (Formula: C₆H₅C(NH)NH₂.HCl, Molecular Weight: 156.6) is a potent inhibitor of serine proteases including thrombine, plasmin, and trypsin, and is also quite suitable for inclusion in the stabilized compositions of this invention.

In general, to provide satisfactory stability for the natriuretic peptide, the sulfonyl fluoride or benzamidine protease inhibitor is employed in an appropriate amount. Thus, compositions of this invention will contain from about 1 pg/mL to about 6000 pg/mL, preferably from about 20 pg/mL to about 2000 pg/mL of the natriuretic peptide and from about 0.01 mM to about 100 mM, preferably from about 0.1 mM to about 10 mM of the sulfonyl fluoride or benzamidine protease inhibitor. Such concentrations of inhibitor are referred to herein as “an effective stabilizing amount.”

The compositions in general are made by combining the natriuretic peptide with the protease inhibitor and other ingredients. While the ingredients may be added or combined in any suitable order, in general, the compositions are made by first preparing a composition containing the protease inhibitor, and then adding the natriuretic peptide.

To manufacture a reference control, processed human or mammalian plasma is spiked with appropriate types and levels of protease inhibitors and antimicrobial agents. The pool is then spiked with BNP and other cardiac risk assessment markers of interest at below, near, and above the clinical decision points for each marker. The pool is then sterile filtered, filled aseptically, and frozen or refrigerated. These steps will be described in the following sections.

The compositions of the invention may contain human or other mammalian blood, serum, plasma, etc., and may be used for testing body fluids obtained from humans as well as from other mammals, e.g. pets, companion animals, mammals in zoological institutions, and other domesticated mammals.

The following examples illustrate the invention as applied to the preparation of controls containing BNP.

Preparation of Base Matrix Using Normal Human Serum:

Units of normal human plasma were pooled and defibrinated according to the procedures known in the prior art. The total protein concentration of the resulting serum base matrix was adjusted to 6.0 g/dL by concentrating the base matrix or diluting it with normal saline solution. The pH of the base matrix was then adjusted to 6.2. Defibrinated plasma was then delipidized according to the procedures known in the prior art to reduce cholesterol and triglyceride levels to <20 mg/dL. This was done in an attempt to improve the optical clarity of the base matrix. The total protein concentration and pH of the resulting base matrix were adjusted to 6.4 g/dL and 6.2, respectively. Enzyme inhibitors, benzamidine and AEBSF, were then added to the base matrix at the final concentration of 9.5 mM and 0.125 mM, respectively. Again, pH was adjusted to 6.2, and the endogenous BNP in the base matrix was then determined using a commercially available assay (Bayer ADVIA Centaur BNP assay). The concentration of the endogenous BNP in a typical preparation of the base matrix was less than 20 pg/mL.

Preparation of the Product:

Stock solutions of BNP and other clinical cardiac risk assessment markers such as troponin I, troponin T, myoglobin, homocysteine, CRP, CK-MB, and NT pro BNP were prepared using native, synthetic, or recombinant materials. Appropriate volumes of the spike solutions were added to the base matrix to prepare a tri-level control to monitor the performance of test procedures for the above analytes at below, near, and above the clinical decision points of the assays. Analyte concentrations were determined after addition of spike solutions, and adjustments to analyte concentrations were made through re-spikes or dilution of the pools to ensure tri-level and clinical utility of the control. The three pools were then aseptically filtered through 0.2 μm filters, later filled in the pre-sterilized small glass vials and closures, and stored at −20 ° C.

Performance of the Product:

Presented in Table 1 are the recovery data for a typical pilot lot of the control. The coefficient of variation (CV) associated with the control is comparable to those obtained from typical patient samples when tested by BNP assays indicating that the control of this invention meets one of the most important characteristics of a quality control material by being as sensitive as the-actual patient sample to all of the anticipated test and analytical variances. This was expected because unlike other controls in the market, the control of this invention does not use an artificial base matrix and instead uses a human serum base matrix. According to the product insert for the Bayer ADVIA Centaur BNP assay, % CVs of 4.7 to 2.9% may be observed at the BNP concentrations ranging from 29.4 to 1736 pg/iL when testing human specimens. According to the product insert for the Abbott AxSYM, this analyzer exhibits %CVs of 6.3 to 4.7% when testing BNP concentrations ranging from 95 to 1587 pg/mL. Furthermore, the results demonstrate that BNP target levels below, near, and above critical/medical decision point of the assays corresponding to the various stages of congestive heart failure can be readily achieved.

TABLE 1
Performance of the Product on Different Test Methods

| | Level 1 | | | Level 2 | | | Level 3 | | |
|---|---|---|---|---|---|---|---|---|---|
| | Mean | | | Mean | | | Mean | | |
| Test Method | pg/mL | SD | % CV | pg/mL | SD | % CV | pg/mL | SD | % CV |
| Bayer Advia Centaur | 108.77 | 1.89 | 1.74 | 463.83 | 4.80 | 1.04 | 1669.32 | 23.32 | 1.40 |
| Abbott AxSYM | 84.92 | 3.04 | 3.58 | 409.65 | 20.38 | 4.97 | 1556.78 | 68.03 | 4.37 |

Closed vial stability of the product was evaluated by using an accelerated stability model to predict product shelf life. For this purpose, vials-of product were stored at an elevated temperature for predetermined periods of time to observe analyte decomposition/degradation more rapidly than the recommended storage temperature of −20° C. and assayed for BNP recovery at the end of various incubation periods. The results of these studies predicted that the product would be stable for at least 3 years when stored unopened at −20° C. The predicted shelf life claim will be supported through the ongoing real time closed vial stability study at −20° C.

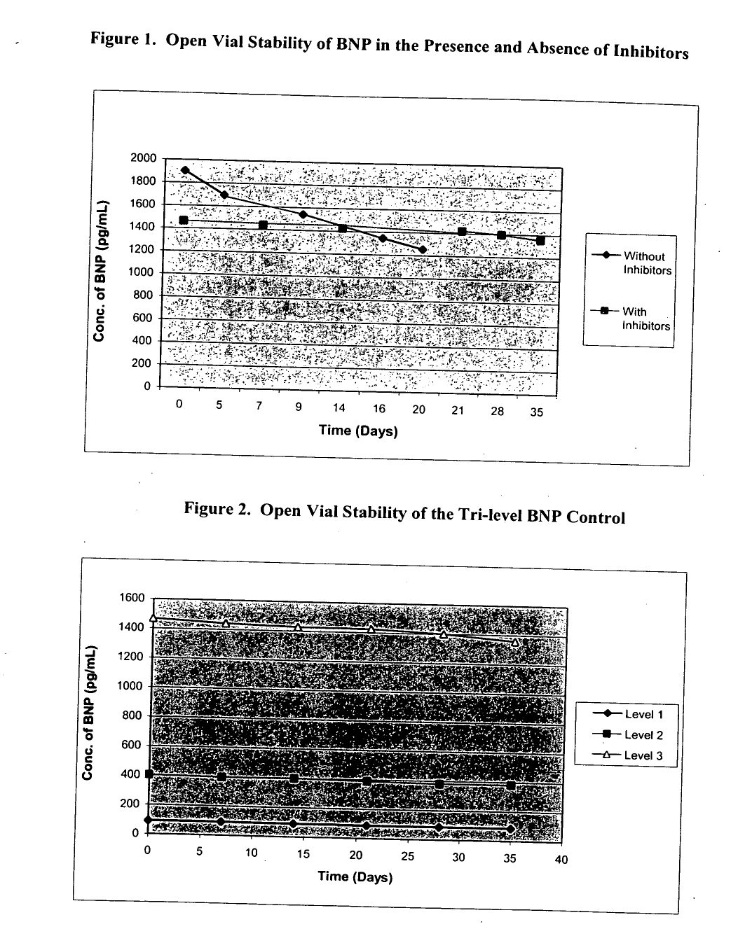

Open vial stability of the product was also evaluated by simulating actual use conditions by the clinicians. This was done by storing the vials at 2-8° C. and removing them from the refrigerator every working day for 35 days, allowing the vials to equilibrate at room temperature for 15 minutes, opening the vials and exposing their contents to the laboratory environment, and closing the vials and returning them to the recommended storage temperature of 2-8° C. Samples of the vials were assayed during this open vial stability study for BNP recovery. Presented in FIG. 1 are the open vial stability results for BNP in pilot lots prepared with and without protease inhibitors. This figure clearly demonstrates the stabilizing effects of the protease inhibitors. Depicted in FIG. 2 are the open vial stability plots for all three levels of the control as a function-of time. The results of this study indicate that the product will be stable for at least 35 days when opened and stored at 2-8° C. An average drop in BNP concentration of 5% was observed during the first 35 days at 2-8° C.

It is understood that the examples and embodiments described herein are for illustrative purposes only and that various modifications or changes in light thereof will be suggested to persons skilled in the art and are to be included within the spirit and purview of this application and scope of the appended claims.

All publications, patents, and patent applications cited herein are hereby incorporated by reference in their entirety for all purposes.

What is claimed is:

1. A stabilized liquid natriuretic peptide composition comprising (a) a natriuretic peptide and (b) an effective stabilizing amount of (i) a protease-inhibiting optionally substituted alkyl or aryl sulfonyl fluoride; or (ii) benzamidine.

2. A composition according to claim 1 wherein the stabilized liquid peptide composition is a reference material for use as a control in a method for determining the level of a natriuretic peptide in a sample.

3. A composition according to claim 1 wherein the natriuretic peptide is a naturally occurring peptide.

4. A composition according to claim 2 wherein the natriuretic peptide is a naturally occurring peptide.

5. A composition according to claim 1 wherein the natriuretic peptide is a synthetic peptide.

6. A composition according to claim 2 wherein the natriuretic peptide is a synthetic peptide.

7. A composition according to claim 1 wherein the natriuretic peptide is a recombinant peptide.

8. A composition according to claim 2 wherein the natriuretic peptide is a recombinant peptide.

9. A composition according to claim 1 wherein the natriuretic peptide is. BNP.

10. A composition according to claim 2 wherein the natriuretic peptide is BNP.

11. A composition according to claim 2 further comprising one or more antimicrobial agents

12. A composition according to claim 2 comprising a body fluid.

13. A composition according to claim 12 wherein the body fluid comprises human or mammalian blood or a human or mammalian blood component.

14. A composition according to claim 13 wherein the body fluid comprises human or mammalian serum or plasma.

15. A composition according to claim 11 wherein the one or more antimicrobial agents are selected from neomycine sulfate, chloramphenicol, and amphotericin.

16. A composition according to claim 1 comprising an effective stabilizing amount of benzamidine.

17. A composition according to claim 2 comprising an effective stabilizing amount of benzamidine.

18. A composition according to claim 1 comprising an effective stabilizing amount of a sulfonyl fluoride.

19. A composition according to claim 2 comprising an effective stabilizing amount of a sulfonyl fluoride.

20. A composition according to claim 18 wherein the sulfonyl fluoride is an optionally substituted alkyl sulfonyl fluoride.

21. A composition according to claim 18 wherein the sulfonyl fluoride is an optionally substituted aryl sulfonyl fluoride.

22. A composition according to claim 19 wherein the sulfonyl fluoride is an optionally substituted alkyl sulfonyl fluoride

23. A composition according to claim 19 wherein the sulfonyl fluoride is an optionally substituted aryl sulfonyl fluoride

24. A composition according to claim 18 wherein the sulfonyl fluoride is selected from (2-aminoethyl)-benzenesulfonyl fluoride, phenylmethanesulfonyl fluoride, 4-amidinophenyl-methanesulfonyl fluoride, 3-acetylbenzenesulfonyl fluoride, 2-aminobenzenesulfonyl fluoride, 3-(3-chlorophenoxyacetamido)benzenesulfonyl fluoride, and peptide aminobenzene sulfonyl fluorides.

25. A composition according to claim 19 wherein the sulfonyl fluoride is selected from (2-aminoethyl)-benzenesulfonyl fluoride, phenylmethanesulfonyl fluoride, 4-amidinophenyl-methanesulfonyl fluoride, 3-acetylbenzenesulfonyl fluoride, 2-aminobenzenesulfonyl fluoride, 3-(3-chlorophenoxyacetamido)benzenesulfonyl fluoride, and peptide aminobenzene sulfonyl fluorides.

26. A composition according to claim 2 further comprising a panel of cardiac risk assessment markers.

27. A composition according to claim 26 wherein the cardiac risk assessment markers are troponin I, troponin T, myoglobin, CK-MB, total CK, homocysteine, pro BNP, NT pro BNP, and hsCRP.

28. A kit for conducting an assay for a natriuretic peptide comprising a composition according to claim 2.

29. A method of preparing a stabilized natriuretic composition comprising combining a natriuretic peptide with an effective stabilizing amount of (i) an optionally substituted alkyl or aryl sulfonyl fluoride or (ii) benzamidine.
